(19) United States
(12) Patent Application Publication
(10) Pub. No.: US 2025/0128034 A1
(43) Pub. Date: Apr. 24, 2025

(54) A MICRO-NEEDLE PRODUCTION METHOD

(71) Applicant: BILKENT UNIVERSITESI ULUSAL NANOTEKNOLOJI ARASTIRMA MERKEZI, ANKARA (TR)

(72) Inventors: ISMAIL ES, ANKARA (TR); ABDULLAH KAFADENK, ANKARA (TR); FATIH INCI, ANKARA (TR)

(73) Assignee: BILKENT UNIVERSITESI ULUSAL NANOTEKNOLOJI ARASTIRMA MERKEZI, ANKARA (TR)

(21) Appl. No.: 18/687,398

(22) PCT Filed: Sep. 6, 2023
(86) PCT No.: PCT/TR2023/050917

§ 371 Date: Feb. 28, 2024

(30) Foreign Application Priority Data
Sep. 6, 2022 (TR) 2022/013871

(51) Int. Cl. A61M 37/00

(52) U.S. Cl. CPC A61M 37/0015; A61M 2037/0046; A61M 2037/0053

(57) ABSTRACT

The present invention relates to a method (100) of producing solid microneedles with a desired geometry, base width, height, height/base width ratio, tip angle and number of needles by means of photolithography and dry etching techniques.

### TECHNICAL FIELD

The present invention relates to a method of producing solid microneedles with a desired geometry, base width, height, height/base width ratio, tip angle and number of needles by means of photolithography and dry etching techniques.

### BACKGROUND OF THE INVENTION

Transdermal drug distribution (TDD) is a technological approach whereby it is ensured that a drug to takes part in the circulation in order that a drug component is distributed throughout different layers of the skin and to achieve a therapeutic result. Although an injection applied by using hypodermic needles is the “gold standard” for TDD, a microneedle-based TID is a new technology whereby drug components are delivered into the bloodstream by means of micron-sized needles. It has been shown that microneedles greatly increase drug permeability by piercing the stratum corneum. While providing a faster recovery at the injection site compared to hypodermic needles, studies on their production are rapidly increasing due to the fact that large molecules can be applied easily without causing pain to the patient. To date, a great number of microneedle-based platforms have been produced by using different materials, mostly silicon and metal. It has been shown that techniques such as dry or wet etching, laser ablation, photolithography and 3D printing, which are used as production phase, are quite effective in the design of these platforms. Microneedles can be produced with a height in the range of 150-1500 μm and a width in the range of 50-250 μm through the use of these methods, but it is limited to realize a production in the desired dimensions between these values.

Therefore, there is a need for a solid microneedle production method with any of geometries in a conical, pyramid, tetrahedron and star structure and whereby parameters such as base width, height, height/base width ratio, tip angle and number of needles can be adjusted.

The United States patent document no. U.S. Pat. No. 6,551,849, an application included in the state of the art, discloses a microneedle production method.

The United States patent document no. US2018161050, another application included in the state of the art, discloses a device which is used to deliver materials or stimuli to targets within the body to produce a desired response.

### SUMMARY OF THE INVENTION

An objective of the present invention is to realize a method of producing solid microneedles having a desired geometry, base width, height, height/base width ratio, tip angle and number of needles by means of photolithography and dry etching techniques.

Another objective of the present invention is to realize a method of obtaining microneedles in a shorter time by optimising the solid microneedle production process.

Another objective of the present invention is to realize a method wherein smoothness of microneedle is eliminated and mechanical strength is enhanced by means of biocompatible metals coated on microneedles.

Another objective of the present invention is to realize a method of producing microneedles in a more economical and reusable way.

### DETAILED DESCRIPTION OF THE INVENTION

“A Microneedle Production Method” realized to fulfil the objectives of the present invention is shown in the figures attached, in which:

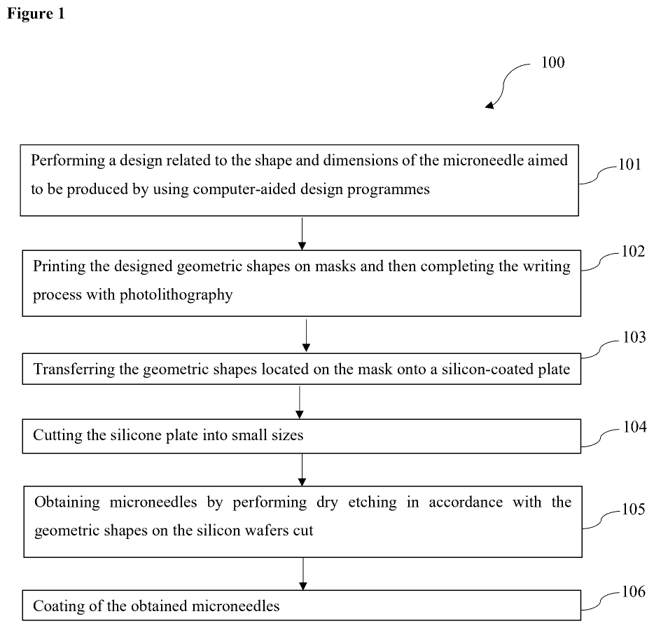

FIG. 1 is a flow chart of the inventive method.

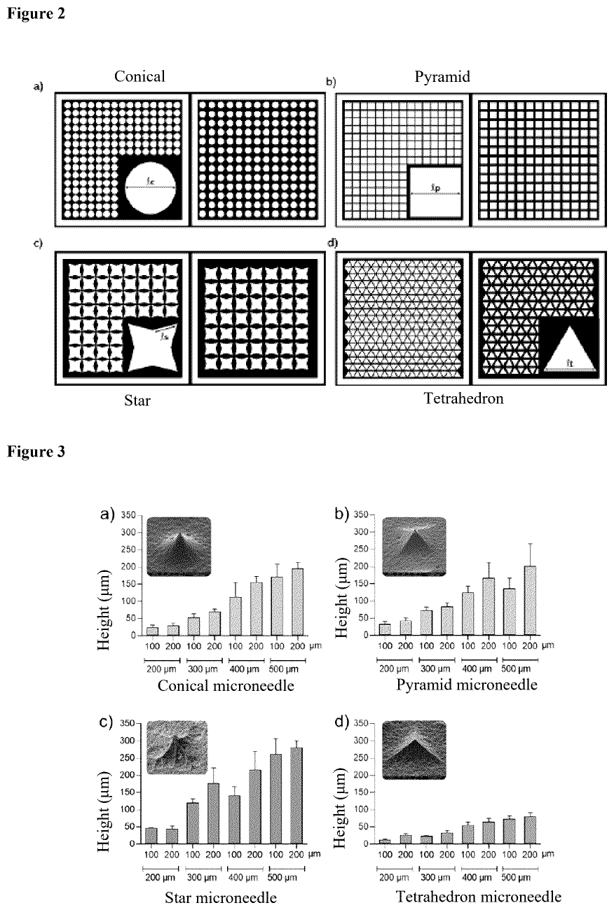

FIG. 2 is a view of the drawings of microneedles produced by the inventive method, in computer-based design programmes.

FIG. 3 is graphs of the average height of (a) conical, (b) pyramid, (c) star and (d) tetrahedron microneedles according to the size of the designed photoresist. (The values on the x-axis (200, 300, 400 and 500 μm) indicate the diameter of the photoresist at the beginning of the process, whereas the other values on the x-axis (100 and 200 μm) are the distance between each of the microneedles).

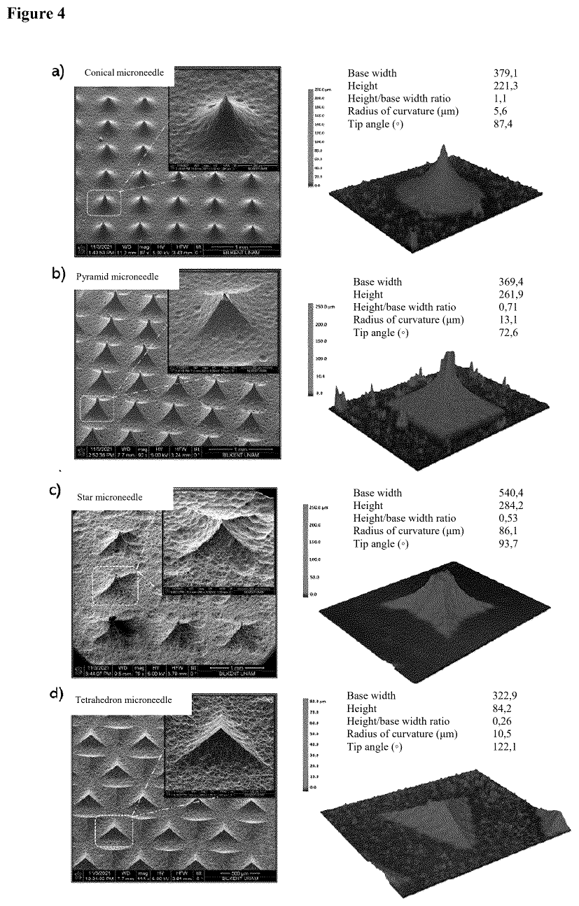

FIG. 4 shows a graph of microneedles after coating analysed by scanning electron microscopy (left) and three-dimensional laser scanning microscopy (right).

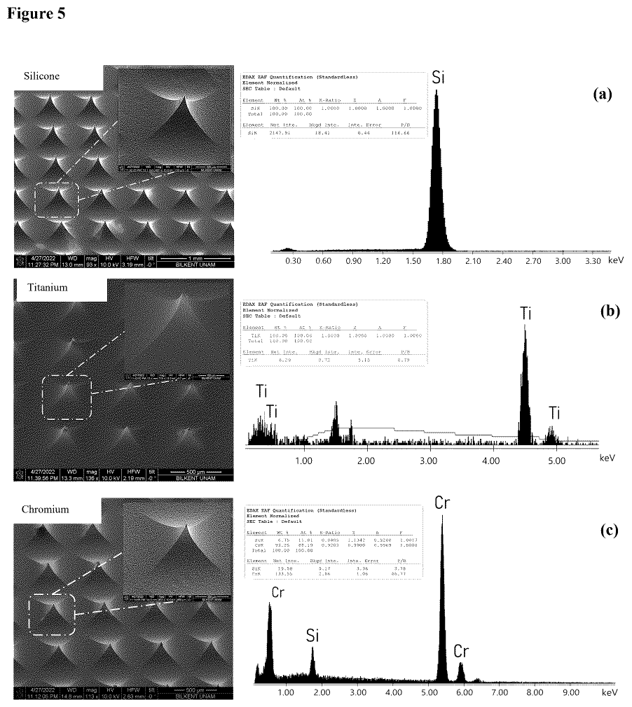

FIG. 5 shows scanning electron microscopy and energy dispersive X-ray analysis results of a) uncoated, b) titanium coated and c) chromium coated microneedles.

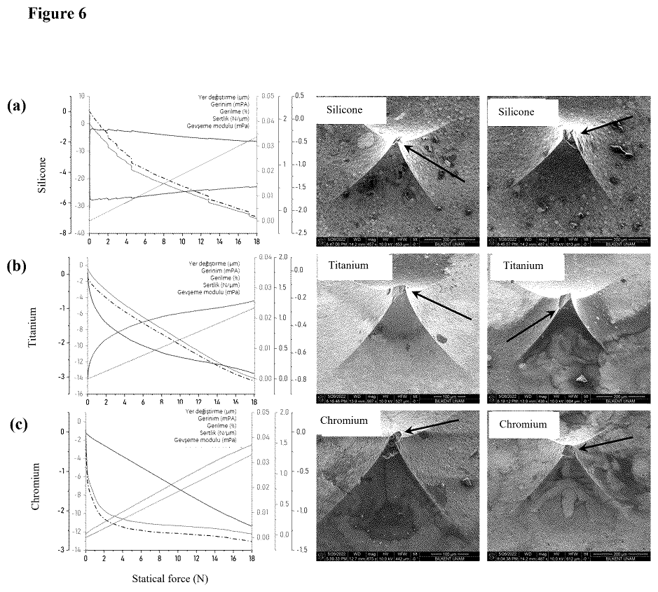

FIG. 6 shows mechanical strength analyses of a) uncoated, b) titanium coated and c) chromium coated microneedles.

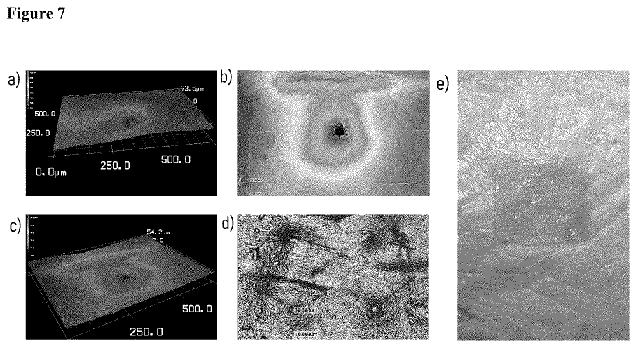

FIG. 7 shows photographs of microneedle punctures on chicken skin taken with a 3D scanning microscope (a-d) and a digital camera (e).

The components illustrated in the figures are individually numbered, where the numbers refer to the following:
100. Method

The inventive method (100) of producing solid microneedles having a desired geometry, base width, height, height/base width ratio, tip angle and number of needles comprises the steps of
performing a design related to the shape and dimensions of the microneedle aimed to be produced by using computer-aided design programmes (101);
printing the designed geometric shapes on masks and then completing the writing process with photolithography (102);
transferring the geometric shapes located on the mask onto a silicon-coated plate (103);
cutting the silicone plate into small sizes (104);
obtaining microneedles by performing dry etching in accordance with the geometric shapes on the silicon wafers cut (105); and
coating of the obtained microneedles (106).

In the step of performing a design related to the shape and dimensions of the microneedle aimed to be produced by using computer-aided design programmes (101) of the inventive method (100): preferably CAD-based design programmes, such as AutoCAD and Layout Editor, are used. In addition, programmes such as Klayout and L-Edit can be used instead of these programmes. The thickness of the silicon wafers used can be determined as 500-1000 μm. In the event that silicon wafers of 1000 μm are used, parameters of a geometrical shape with conical, pyramid, tetrahedron, star and polyhedron structures; base width in the range of 100-500 μm; height in the range of 50-800 μm; height/base width ratio in the range of 0.1-8; tip angle <90° and needle number in the range of 50-2000 needles/cm² are determined related to the microneedle to be produced by means of CAD-based design programmes (Table 1). The geometrical shapes designed by the design programmes related to the microneedles are shown in the FIG. 2.

TABLE 1
Design parameters of microneedles determined in AutoCAD

| | | DISTANCE [symbol] | | | |
|---|---|---|---|---|---|
| | | BETWEEN [symbol] | NUMBER [symbol] | | |
| TYPE OF [symbol] | PHOTORESIST [symbol] | MICRO [symbol] | OF MICRO [symbol] | THE AREA COVERED [symbol] | THE AREA [symbol] |
| MICRO- [symbol] | EDGE [symbol] | NEEDLES [symbol] | NEEDLES [symbol] | BY MICRONEEDLES [symbol] | AVAILABLE FOR [symbol] |
| NEEDLE [symbol] | (μm) | (μm) | IN 1 CM² [symbol] | ( [symbol] ) | ABRASION mm2 [symbol] |
| CONICAL | 300 | 100 | 900 | 0.03142 | 58. [symbol] |
| [symbol] | | 200 | 529 | 0.03142 | [symbol] |
| | 300 | 100 | [symbol] | [symbol] | 52.09014 |
| | | 200 | 324 | [symbol] | 63.40054 |
| | 400 | 100 | 324 | 0.12566 | [symbol] |
| | | 200 | 225 | 0.12566 | [symbol] |
| | 500 | 100 | 225 | [symbol] | [symbol] |
| | | 100 | 169 | [symbol] | [symbol] |
| PYRAMID | 200 | 100 | 900 | 0.04 | [symbol] |
| [symbol] | | 200 | 529 | 0.04 | [symbol] |
| | 300 | 100 | 484 | 0.09 | [symbol] |
| | | 200 | 324 | 0.09 | [symbol] |
| | 400 | 100 | 378 | 0.16 | [symbol] |
| | | 200 | 225 | 0.16 | [symbol] |
| | 500 | 100 | 225 | 0.25 | 30.0541 |
| | | 200 | 169 | 0.25 | 44.0541 |
| TETRAHEDRON | 200 | 100 | 1452 | [symbol] | [symbol] |
| [symbol] | | 200 | 720 | [symbol] | 73.8337 |
| | 300 | 100 | 900 | [symbol] | [symbol] |
| | | 200 | 520 | [symbol] | [symbol] |
| | 400 | 100 | 580 | [symbol] | [symbol] |
| | | 200 | [symbol] | [symbol] | 61.91754 |
| | 500 | 100 | [symbol] | [symbol] | [symbol] |
| | | 200 | 266 | [symbol] | [symbol] |
| STAR | 200 | 100 | 324 | [symbol] | [symbol] |
| [symbol] | | 200 | 225 | [symbol] | [symbol] |
| | 300 | 100 | 169 | [symbol] | [symbol] |
| | | 200 | 121 | [symbol] | [symbol] |
| | 400 | 100 | 100 | [symbol] | [symbol] |
| | | 200 | 81 | [symbol] | [symbol] |
| | 500 | 100 | 64 | [symbol] | [symbol] |
| | | 200 | 49 | [symbol] | [symbol] |

[symbol]

indicates data missing or illegible when filed

In the step of printing the designed geometric shapes on masks and then completing the writing process with photolithography (102) of the inventive method (100); the microneedle shapes drawn in the design programmes are printed on chromium-structured masks or acetate paper by using a laser mask printer. Apart from that, the direct-printing method can be used to print on a silicone sheet without using any mask. In order to enhance the photoresist durability during the writing process, silicon nitrate (Si₃N₄)—which has no or very low selectivity to XeF₂— is coated and printing is completed by photolithography. Metals, metal oxides and metal nitride materials can also be used instead of Si₃N₄.

In the step of transferring the geometric shapes located on the mask onto a silicon-coated plate (103) of the inventive method (100); the geometric shapes located on the mask are transferred onto a silicon structured plate coated with a photoresist with a thickness of 1-2 μm for UV mask printing. AZ-based or SU-8-based materials can be used as photoresist. Depending on the model of the photoresist used, a coating of 1 μm-100 μm can be applied on the silicon wafer.

In the step of cutting the silicone plate into small sizes (104) of the inventive method (100); the silicon wafer whereon the desired geometrical structures are located is cut with a micro saw into any of the square, rectangular, round, triangular or polygonal shapes, preferably 0.5-10 cm².

In the step of obtaining microneedles by performing dry etching in accordance with the geometric shapes on the silicon wafers cut (105) of the inventive method (100): each silicon wafer cut into 1 cm² squares is subjected to isotropic dry etching with XeF₂ (xenon fluoride) gas and the designed microneedles are obtained as a result of etching. The pressure of XeF₂ gas during etching is optimized so as to be 1-4 m Torr and the etching time is optimized so as to be 10-900 s.

In the step of coating of the obtained microneedles (106) of the inventive method (100); the microneedles are coated with metals in the form of chromium, titanium, stainless steel, aluminium, copper, nickel, zircon and molybdenum, or with materials which are neither metallic nor organic in the form of ceramics, in order to increase their robustness against mechanical stress and to eliminate surface smoothness.

In the inventive method (100), the formation time of microneedles is varied according to the drawings designed in CAD and the width of the area outside the photoresist. According to the preliminary calculation carried out in the CAD programme, it was calculated that an initial etching area of approximately 60 mm² per microneedle for ˜40 microneedles of 1 cm² each is required for the formation of microneedles at the desired height with an etching time of 30 seconds and a gas pressure of 4 mTorr, which requires ˜12 hours. Microneedle formation was performed via a XeF₂ selective etching device (SPTS Technologies, UK).

The microneedles obtained by the inventive method (100) were characterised. For this purpose, microneedles were examined under a light microscope immediately at first after the dry etching process. After it was determined that the microneedles acquired the desired shape, the etching process was terminated. The surface characteristics such as morphological and roughness (Table 2) of the microneedles, which were determined to be free of photoresist residues, were determined by nano-scanning electron microscopy and 3D laser scanning microscopy (Keyence vk ×100) (FIGS. 3 and 4). The height and width of the microneedles were also determined by using these microscopes. Whether the microneedles were coated with chromium and titanium to increase their mechanical strength was determined by EDX analysis (FIG. 4).

TABLE 2
Changes in surface roughness of microneedles
before and after coating

| Coating Process | Geometry | Ra* | Rz** | Rq*** |
|---|---|---|---|---|
| Uncoated | Pyramid | 0.71 | 4.671 | 0.888 |
| | Conical | 0.585 | 3.567 | 0.712 |
| Titanium Coating | Pyramid | 0.458 | 2.366 | 0.541 |
| | Conical | 0.475 | 2.537 | 0.583 |
| Chromium Coating | Pyramid | 0.379 | 1.677 | 0.450 |
| | Conical | 0.351 | 1.757 | 0.415 |

With the inventive method (100), a solid, i.e. non-perforated, microneedle is produced so as to be used for transdermal drug release. Non-perforated microneedles are generally used in the pharmaceutical and cosmetic industry to open micro-structured pores on the skin and thus to facilitate the penetration of substances such as drugs or creams under the skin. By means of the optimized process, microneedles were produced by using the dry etching technique in less time and using the most suitable xenon difluoride gas pressure. In addition, the drawings that can be used according to the geometrical characteristics and number of the targeted microneedle have been optimized and made ready for production. The effect of generally biocompatible metals, such as chromium and titanium coated on microneedles, on the roughness of the microneedle was determined and their mechanical durability was increased. The roughness obtained after coating is below 1 μm. The coating was performed by Magnetron Sputter Coating technique. Apart from this technique, coating ion-beam evaporation or diamond-like carbon (DLC) techniques can be used as well. Microneedles produced in this way can be reused and they have become economically attractive especially for the pharmaceutical, cosmetics, agriculture and food sectors.

Within these basic concepts; it is possible to develop various embodiments of the inventive “Microneedle Production Method (100)”; the invention cannot be limited to examples disclosed herein and it is essentially according to claims.

1. A method (100) of producing solid microneedles having a desired geometry, base width, height, height/base width ratio, tip angle and number of needles; characterized by the steps of
performing a design related to the shape and dimensions of the microneedle aimed to be produced by using computer-aided design programmes (101);
printing the designed geometric shapes on masks and then completing the writing process with photolithography (102);
transferring the geometric shapes located on the mask onto a silicon-coated plate (103);
cutting the silicone plate into small sizes (104);
obtaining microneedles by performing dry etching in accordance with the geometric shapes on the silicon wafers cut (105); and
coating of the obtained microneedles (106).

2. A method (100) according to claim 1; characterized in that in the step of performing a design related to the shape and dimensions of the microneedle aimed to be produced by using computer-aided design programmes (101); CAD-based design programmes such as AutoCAD, Layout Editor, Klayout and L-Edit are used.

3. A method (100) according to claim 1; characterized in that in the step of performing a design related to the shape and dimensions of the microneedle aimed to be produced by using computer-aided design programmes (101); the thickness of the silicon wafer used is determined as 500-1000 μm.

4. A method (100) according to claim 1; characterized in that in the step of performing a design related to the shape and dimensions of the microneedle aimed to be produced by using computer-aided design programmes (101); parameters of a geometrical shape with conical, pyramid, tetrahedron, star and polyhedron structures; base width in the range of 100-500 μm; height in the range of 50-800 μm; height/base width ratio in the range of 0.1-8; tip angle <90° and needle number in the range of 50-2000 needles/cm2 are determined related to the microneedle to be produced by means of CAD-based design programmes.

5. A method (100) according to claim 1; characterized in that in the step of printing the designed geometric shapes on masks and then completing the writing process with photolithography (102); the microneedle shapes drawn in the design programmes are printed on chromium-structured masks or acetate paper by using a laser mask printer.

6. A method (100) according to claim 1; characterized in that in the step of printing the designed geometric shapes on masks and then completing the writing process with photolithography (102); the microneedle shapes drawn in the design programmes are printed on a silicone sheet without using any mask, by using direct-printing method.

7. A method (100) according to claim 5; characterized in that in the step of printing the designed geometric shapes on masks and then completing the writing process with photolithography (102); in order to enhance the photoresist durability during the writing process, silicon nitrate (Si3N4)—which has no or very low selectivity to XeF2- is coated and printing is completed by photolithography.

8. A method (100) according to claim 7; characterized in that in the step of printing the designed geometric shapes on masks and then completing the writing process with photolithography (102); materials such as metals, metal oxides and metal nitride materials can be used instead of Si3N4.

9. A method (100) according to claim 1; characterized in that in the step of transferring the geometric shapes located on the mask onto a silicon-coated plate (103); the geometric shapes located on the mask are transferred onto a silicon structured plate coated with a photoresist with a thickness of 1-2 μm for UV mask printing.

10. A method (100) according to claim 9; characterized in that in the step of transferring the geometric shapes located on the mask onto a silicon-coated plate (103); AZ-based or SU-8-based materials are be used as photoresist.

11. A method (100) according to claim 10; characterized in that in the step of transferring the geometric shapes located on the mask onto a silicon-coated plate (103); depending on the model of the photoresist used, a coating of 1 μm-100 μm is made on the silicon wafer.

12. A method (100) according to claim 10; characterized in that in the step of cutting the silicone plate into small sizes (104); the silicon wafer whereon the desired geometrical structures are located is cut with a micro saw into any of the square, rectangular, round, triangular or polygonal shapes of 0.5-10 cm2.

13. A method (100) according to claim 1; characterized in that in the step of obtaining microneedles by performing dry etching in accordance with the geometric shapes on the silicon wafers cut (105); each silicon wafer cut into 1 cm2 squares is subjected to isotropic dry etching with XeF2 gas and the designed microneedles are obtained as a result of etching.

14. A method (100) according to claim 1; characterized in that in the step of obtaining microneedles by performing dry etching in accordance with the geometric shapes on the silicon wafers cut (105); the pressure of XeF2 gas during etching is optimized so as to be 1-4 m Torr and the etching time is optimized so as to be 10-900 s.

15. A method (100) according to claim 1; characterized in that in the step of coating of the obtained microneedles (106); the microneedles are coated with metals in the form of chromium, titanium, stainless steel, aluminium, copper, nickel, zircon and molybdenum, or with materials which are neither metallic nor organic in the form of ceramics, in order to increase their robustness against mechanical stress and to eliminate surface smoothness.
